(19) United States
(12) Patent Application Publication
(10) Pub. No.: US 2007/0129613 A1
(43) Pub. Date: Jun. 7, 2007

(54) Flexible light sources and detectors and applications thereof

(75) Inventors: Katie Rochester, Worcestershire (GB); Ian Charles Sage, Worcestershire (GB); Tej Paul Kaushal, Worcestershire (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Qinetiq Limited

(21) Appl. No.: 10/579,315

(22) PCT Filed: Nov. 18, 2004
(86) PCT No.: PCT/GB04/04871

§ 371 Date: May 16, 2006

(30) Foreign Application Priority Data
Nov. 18, 2003 (GB) 0326821.6

(51) Int. Cl. A61B 5/00

(52) U.S. Cl. 600/310; 600/322

(57) ABSTRACT

Flexible and conformal medical light sources and related diagnostic devices directed to monitoring blood characteristics (e.g. levels of CO, oxygen, or bilirubin) and photo-therapeutic devices for treatment of ailments such as psoriasis and some forms of cancer. The flexible light source preferably comprises one or more organic light emitting diodes on a flexible substrate. Light sources may also be used for purposes of treatment. The substrate can also form a integral strap for attachment of the device over or around the patient's body. Optionally, the device comprises a photo-detector arranged to detect and monitor emissions from the sources. Flexible and conformal medical light detectors and devices are also provided.

### FIELD OF THE INVENTION

The present invention relates to optoelectronic devices in general and in particular to flexible light sources (for example organic light emitting diodes) and detectors, and applications thereof. Applications include, but are not limited to, use in medical applications including therapeutic light sources and patient monitoring equipment.

### BACKGROUND TO THE INVENTION

The use of light sources for medical purposes is well known, and may be broadly categorised into use for monitoring purposes and use for therapeutic purposes.

For monitoring purposes it is well-known to use light sources in monitoring devices which take advantage of the absorption spectrum of various blood constituents to facilitate non-intrusive detection of human and animal patient blood characteristics.

One such device is the pulse oximeter, and such devices have been in common use in hospital operating theatres since the 1970's. In more recent years such devices have seen widespread use in other situations, including use in post-operative monitoring, during patient transport, on general wards, and for monitoring of premature or small infants. Neonatal monitoring is an important application of pulse oximetry since premature infants may have periods of apnoea and require extra oxygen. Conversely, it is also important not to oversaturate infants with oxygen. Other medical applications of pulse oximeters include monitoring of aircraft pilots during flight, particularly at altitude where blood oxygen levels may become abnormal, and others operating in environments which may adversely affect blood oxygen levels.

Known pulse oximeters comprise a sensor having a light source and a photodetector. In known oximeters the sensors comprise solid state photodiodes and light emitting diodes (LEDs) to measure light absorption through tissue, typically via a sensor attached to the finger, toe, hand, or foot of the individual to be monitored. Two wavelengths of light—in the red and near infra-red (NIR) spectrum respectively—are emitted in a time-interleaved manner, typically by two adjacent LEDs, with a shared photodiode arranged to detect emissions from each in turn. By measuring the difference in intensity of light received from each LED, a measure of blood oxygen content may be derived by known means.

Some known sensors are manufactured in sizes especially for babies. However even these are far too large for premature and small babies, who need intensive monitoring. These sensors use LEDs which are incorporated into a foam or self-adhesive wrap.

However, referring to FIG. 1(a), a known problem with such sensors is that known LEDs 73 are made inside rigid glass or plastic cases which significantly limits the curvature of the sensor device achievable when applying the oximeter to the patient 61. In some cases it is also difficult to achieve good optical contact between the sensor components and the patient's skin owing to the undesirably large size and inflexibility of the sensor components. Since such sensors cannot, for example, closely follow the tight skin curvature of a tiny baby, the sensors are prone to becoming detached or moving with respect to the patient during use and may thereby give rise to false alarms.

A further well-known problem associated with existing oximeters, and similar sensors, is the so-called “penumbra effect”. This arises when the respective paths between the multiple light sources and the detector differ significantly. Because known LEDs are discrete rigid devices and effectively provide point sources of light, they cannot typically be sufficiently closely located adjacent one another to ensure that the respective paths to the detector are consistently sufficiently close when the device is actually applied to the patient. Consequently this adds to the difficulties in siting the sensors on a patient and the potential uncertainty of the readings obtained.

Other similar devices are known for monitoring blood characteristics including bilirubin and carbon monoxide (CO) levels. In such devices three or more sources of light at distinct wavelengths are employed so that, in general, two, three, or more are employed according to the characteristic to be monitored.

The rigid nature of the electronic components of existing sensors means that the sensor's carrying strip 71 does not follow well the patients' contours. This problem is partially overcome in known oximeters by the use of self-adhesive strips in which the carrying strip adheres to the patient to avoid rocking and slippage. However, the use of self-adhesive strips has the undesirable side-effect of causing skin irritation in some cases—particularly in young babies—and such strips must therefore be re-sited frequently (for example every 3-4 hours). As a result, the adhesive on the sensor quickly becomes degraded and no longer sticky typically after only a single day's use. Known sensors are sufficiently large as to cover a relatively large area of the patient when in place. This is particularly so in the case of small babies. Because of this, such sensors are often applied over the foot, even when this site is not otherwise ideal for monitoring the patient, whether medically or for the patent's comfort.

Hook-and-loop fastenings (for example Velcro™) are well known as a simple and rapid general-purpose fastening and unfastening means. However the lack, in known sensors, of a snug fit around the patient—owing at least in part to the rigid nature of some component parts—means that use of such fastening means alone in known sensors in place of self-adhesion would lead to an arrangement in which the electronic components would be prone to rocking or slipping around the patient. This in turn would give rise to inaccurate readings and ultimately to false alarms were the oximeter to loosen or detach entirely from the patient. If an adhesive strip is, as in known sensors, used in this way there is no need to employ additional attachment means (for example hook-and-loop means) to fasten the strip to itself since attachment to the patient obviates such additional fastening means.

Turning now to therapeutic light sources, it is known to employ phototherapy for skin conditions including, but not limited to, psoriasis. In the case of psoriasis, light in the ultra-violet (UV) spectrum is utilised in treatment. Patients are given a sensitising agent (in tablet or cream form) which acts to sensitise part or all of the patient to UVA radiation (320-400 nm). The patient is then exposed for a time to this wavelength of light by means of a UVA lamp. Exposure is repeated as necessary until treatment is completed. Known light sources are in the form of a conventional UVA lamp located at a moderate distance from the patient and oriented to illuminate the area to be treated. Consequently, some parts of the body may be exposed which do not require specific treatment and, since light from the source is dissipated widely, the available light is also not efficiently directed to the area to be treated.

Unfortunately, and particularly in the case where the patient has taken the sensitsing agent in tablet form rather than applying the cream to the affected area to be treated, there is an associated danger of eye damage arising from inadvertent exposure of the eyes to the UVA lamp during treatment. Where the skin condition is widespread, it may nevertheless be more appropriate to introduce the sensitising agent in tablet form and to take physical precautions (for example a UVA-proof blindfold) to protect the eyes.

In photodynamic therapy, patients are injected with special dyes, which then accumulate in tumour sites. The tumour sites are then irradiated with light at a predetermined wavelength (typically in the red spectrum) which is absorbed by the dyes, resulting in damage to tumour cells where the dye has accumulated.

Organic Light Emitting Diodes (OLEDs) are known in the art and typically comprise a light emitting layer sandwiched between an anode and a cathode. Typically the anode is in contact with a transparent substrate, the anode itself typically being semi-transparent.

Known uses of such OLEDs include thin displays—suitable for computer displays, cellular phones, video cameras, etc.—which may be flexible in nature. Such displays must, by their very nature, comprise a relatively large array of small discrete OLEDs, with potentially one or more OLEDs corresponding to a single pixel, in order to display the required the text or images. The greater the resolution required the greater the number of OLEDs. Multiple OLEDs per pixel are required for colour displays, each OLED per pixel providing complementary colour output so as in combination to achieve a full-colour display. Such displays are often referred to as “paper-like” in that they are both thin and flexible. Clearly, the OLEDs used in this way must emit in the visible spectrum and their emissions are intended to be viewed, either directly or indirectly.

Use of organic photo detectors is known in devices such as, for example, photocopiers and laser printers. In such arrangements the organic photo-detector is applied to a rigid surface in the form of a drum formed typically of metal (for example aluminium). A layer over the photo-detector, having low electrical conductivity in the dark, is given a static electrical charge by means of a corona wire. By allowing light—typically in the blue region of the spectrum—to fall in a predetermined pattern onto the photo-detector layer, the electrical charge within the illuminated areas is discharged leaving the charge only on the unilluminated areas. When toner is subsequently applied to the drum, it attaches only to the charged areas, from which it is conveyed to the printing paper. One photo-detecting compound used for photocopier drums is Titanyl Pthalocyanine (TioPC).

U.S. Pat. No. 4,111,850 describes a carbazole based organic photoconductor fabricated specifically on a flexible substrate. However this is designed to detect in the UV spectrum, and although it describes dopants to extend the sensitivity into the visible, these would be unsuitable for detection of red or near infra-red (NIR).

U.S. Pat. No. 4,167,331 discloses methods of analysing signals from pulse oximeters and other sensors in which light of two different wavelengths is passed through or reflected from a member of the body so as to be modulated by pulsatile blood flow therein. The amplitudes of the alternating current components of the logarithms of the respective light modulations are compared by taking their molecular extinction coefficients into account so as to yield the degree of oxygen saturation. By adding a third wavelength of light, the percentage of other absorbers in the blood stream such as a dye or carboxyhemoglobin can be measured. Fixed absorbers reduce the amount of light that passes through or is reflected from the body member by a constant amount and so have no effect on the amplitudes of the alternating current components that are used in making the measurements.

U.S. Pat. No. 5,685,299 discloses a further technique for analysing the signals output by similar sensors.

U.S. Pat. No. 6,555,958 describes a method of utilising phosphor to down-convert ultra-violet emissions from LEDs to the blue/green emissions. U.S. Pat. No. 5,874,803 describes use of a filter/phosphor stack to down-convert from blue wavelengths emitted by OLEDs to red/green wavelengths. In both cases down-conversion is to the visible spectrum.

### SUMMARY OF THE INVENTION

The present invention provides flexible and conformal medical light sources and detectors and associated diagnostic devices directed to monitoring blood characteristics (e.g. levels of CO, oxygen, or bilirubin) and photo-therapeutic devices for treatment of ailments such as psoriasis and some forms of cancer. The invention is intended for use both on the human and animal body.

According to a first aspect of the present invention there is provided a medical light source comprising one or more flexible light emiting diodes formed upon respective regions of flexible substrate.

The flexible light emitting diodes may be formed upon a single flexible substrate.

The medical light source may be arranged to be sufficiently flexible to permit the light source, in operation, to conform to a portion of the body of a patient to which light from the light source is to be applied.

Advantageously, a closer and more stable fit can be provided to the patient's body.

The flexible light emitting source may comprise an organic light emitting diode. However other flexible light emitting sources may be employed including, for example, those employing porous silicon structures.

The flexible light emitting diode may emit light at a wavelength suitable for diagnosis or therapy of a medical condition of the human or animal body.

In some embodiments flexible light emitting diode emits light in the red to infra-red region of the spectrum.

In some embodiments the flexible light emitting diode emits light in the near infra-red region of the spectrum.

In some embodiments the flexible light emitting diode emits light in a non-visible region of the spectrum.

The medical light source may comprise a plurality of flexible light emitting diodes arranged to emit light at mutually distinct wavelengths.

The medical light source may comprise at least two light emitting diodes arranged to emit at mutually distinct wavelengths, the light emitting diodes being arranged such that light at those distinct wavelengths is emitted substantially evenly across the sum of the areas defined by the light emitting diodes emitting at those wavelengths.

The medical light source may comprise a photo-detector arranged, in operation, to detect light emitted from the one or more flexible light emitting diodes.

The medical light source may comprise a strap comprising attachment means for attachment of the medical light source around or to a patient's body.

The flexible substrate may form the strap.

The attachment means may be one of hook-and-loop means, barb-and-slot means, and self-adhesive means.

The light emitting diode may comprise a triplet emitter.

The light emitting diode may comprise one or more components arranged to wavelength-shift light emitted within the light source from a first wavelength to a second wavelength.

The medical light source may comprise a fluorescent emitter and in which wavelength-shifting is at least partially achieved by means of a fluorescent emitter.

The medical light source may comprise a wavelength-shifting grating and in which wavelength-shifting is at least partially achieved by means of the wavelength-shifting grating.

The medical light source may comprise a micro-cavity and in which wavelength-shifting is at least partially achieved by means of the micro-cavity.

The second wavelength may be determined by tuning of the micro-cavity.

The micro cavity may be tuned to emit light at a third wavelength substantially perpendicular to the plane of the light emitting diode.

According to a second aspect of the present invention there is provided a medical sensor comprising one or more flexible photodetectors formed upon respective regions of flexible substrate.

The medical light sensor may be arranged to be sufficiently flexible to permit the photodetector, in operation, to conform to a portion of the body of a patient.

The medical sensor may also comprise a medical light source according to the first aspect and at least one of the one or more flexible photodetectors may be arranged so as, in operation, to detect light emitted by at least one of the flexible light emitting diodes.

The medical sensor may comprise two or more flexible light emitting diodes arranged to emit light on a time-interleaved basis.

The medical sensor may comprise a plurality of the medical light sources arranged, in operation, to emit light at wavelengths suitable for diagnosis of levels of at least one of oxygen, carbon monoxide, and bilirubin in a human or animal body.

The light detector may be an organic photovoltaic detector.

According to a further aspect of the present invention there is provided a method of operating a medical light source according to the first aspect in a pulsed mode having a predetermined pulse period, such that the triplet emitter is activated for a period calculated to ensure that emissions fall to acceptable levels before a subsequent light pulse is emitted.

The predetermined pulse period may be less than or equal to 25 ms.

Timing of emitted light pulses may be determined responsive to an indication of the pulse timing of a patient to which the sensor is applied.

According to a further aspect of the present invention there is provided an organic light emitting diode arrangement comprising an organic light emitting diode arranged to emit light in a visible region of the spectrum and a wavelength-converting layer arranged to convert visible emissions from the organic light emitting diode to emissions in the infra-red region of the spectrum.

According to a further aspect of the present invention there is provided an organic light emitting diode arrangement comprising an organic light emitting diode arranged to emit light in the blue region of the spectrum and a wavelength-converting layer arranged to convert blue emissions from the organic light emitting diode to emissions in the infra-red region of the spectrum.

Green emissions may similarly be converted to infra-red emissions.

The wavelength-converting layer may comprise a phosphor based compound.

The wavelength-converting layer may comprise an infra-red edge filter.

According to a further aspect of the present invention there are provided organic light emitting diodes suitable for use in medical light sources.

According to a further aspect of the present invention there is provided organic photovoltaic detectors suitable for use in medical sensors.

The invention is also directed to methods by which the described apparatus operates and can be operated and including method steps for carrying out every function of the apparatus.

According to further aspects of the invention there are provided organic light emitting diodes (including wavelength-shifting OLEDs) and photovoltaic detectors, all of which are suitable for use in medical light sources in general and for medical sensors (including pulse oximeters and similar devices) in particular.

The preferred features may be combined as appropriate, as would be apparent to a skilled person, and may be combined with any of the aspects of the invention. Other advantages of the invention, beyond the examples indicated above, will also be apparent to the person skilled in the art.

### BRIEF DESCRIPTION OF THE DRAWINGS

In order to show how the invention may be carried into effect, embodiments of the invention are now described below by way of example only and with reference to the accompanying figures in which:

FIG. 1(a) shows a schematic diagram of an example of a sensor according to the prior art;

FIG. 1(b) shows a schematic diagram of an example of a sensor in accordance to the present invention;

FIGS. 2(a)-2(c) show schematic diagrams of the structures of three examples of organic light emitting diodes in accordance with the present invention;

FIGS. 3(a)-(c) show a schematic graphs of wavelength shifting of OLED emissions in accordance with the present invention.

FIG. 4 shows a schematic diagram of the structure of a further example of an organic light emitting diode in accordance with the present invention;

FIGS. 5(a)-5(e) show schematic diagrams of the structures of example photo-detectors in accordance with the present invention;

FIGS. 6(a) and 6(b) show schematic diagrams of a first sensor arrangement in accordance with the present invention;,

FIG. 7 shows a schematic diagram of a sensor according to the present invention in operation;

FIG. 8 shows a schematic diagram of a second sensor arrangement in accordance with the present invention;

FIG. 9 shows an example of a therapeutic light source in accordance with the present invention;

FIG. 10 shows a schematic diagram of a therapeutic light source according to the present invention in operation.

FIGS. 11(a)-11(e) show schematic diagrams of flexible light source layouts in accordance with the present invention;

### DETAILED DESCRIPTION OF THE INVENTION

The present inventors have identified that the use of flexible LEDs (for example organic LEDs or polymer based light sources, formed upon flexible substrates) as medical light sources offers many advantages over known light sources for diagnostic and therapeutic purposes.

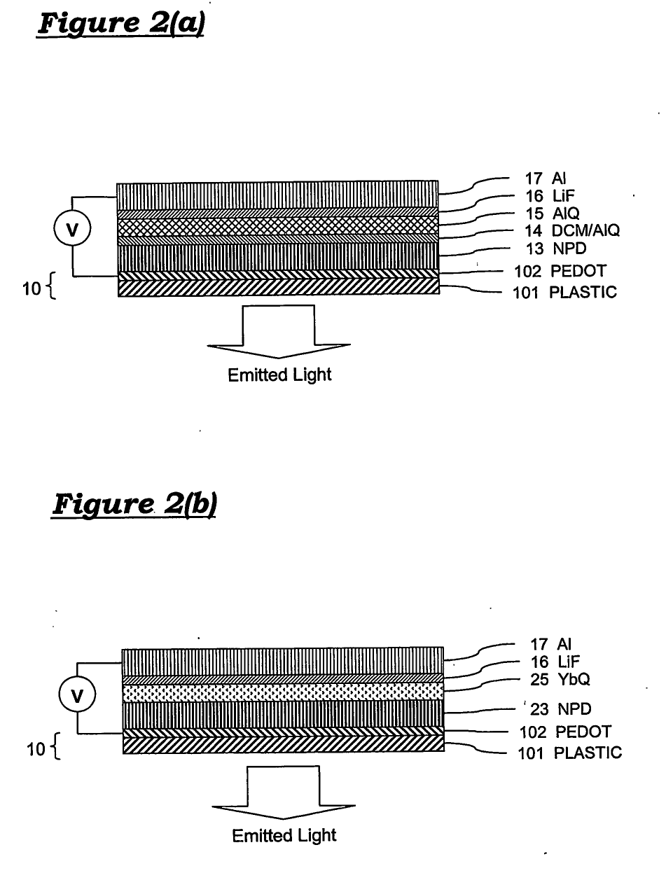

Referring to FIG. 2(a), a first embodiment of a flexible organic light emitting diode is formed upon a plastic substrate 10, which may be approximately 50 mm long and 13 mm wide. ORGACON™ flexible substrate (AGFA) may be used. ORGACON is a commercially available PET (Poly Ethylene Terephthalate) film 101 coated with a conductive polymer (PEDOT/PSS—Polyethylene-Dioxythiophene in Polystyrenesulphonic acid) 102. Several varieties of ORGACON are available, of which a preferred variety provides a substrate which is 125 microns thick and has sheet resistance of 350 ohms/square. The OLED is formed upon the substrate by forming successive layers as follows.

Further layers are then evaporated onto the flexible substrate to form a red-emitting. OLED:
a 60 nm layer 13 of NPD (N,N′-diphenyl- N,N′-bis(1-napthylphenyl)-1,1′-biphenyl-4,4′-diamine);
a 30 nm layer 14 of AlQ (Aluminium 8-hydroxyquinolinate) coevaporated with DCM2 (4-Dicyanomethylene-2-methyl-6-[2-(2,3,6,7-tetrahydro-1H,5H-benzo[l,j]-quinolozin-8-yl)-vinyl]-4H-pyran) laser dye at 2% concentration;
a 30 nm 15 layer of AlQ
a 0.6 nm layer 16 of Lithium Fluoride (LiF); and
a 150 nm layer 17 of Aluminium to act as cathode.

The resulting red-emitting OLED emits light at approximately 616 nm, corresponding to the emission peak expected from DCM2 laser dye.

Whilst the present specific embodiment uses a substrate of PEDOT and PET, it will be apparent to the person skilled in the art that other flexible substrates could be used including, for example, Indium Tin Oxide (ITO) coated PET from Sheldahl with sheet resistance of 60 ohms/square and PET thickness 150 microns.

In the first embodiment of a near infra-red (NIR) emitting OLED shown in FIG. 2(b), a solution of Ytterbium Chloride was mixed with a solution of 8-hydroxyquinoline to form a powder (known as YbQ) which was washed, dried and sublimed. An OLED is then constructed with the following layering structure:
a 68 nm layer 23 of NPD;
a 38 nm layer 25 of YbQ;
a 0.6 nm layer 16 of LiF; and
a 150 nm layer 17 of Aluminium to act as cathode.

The resulting device emits light at the main Ytterbium transition line of 980 nm.

Referring now to FIG. 2(c), a second, preferred, embodiment of a NIR-emitting OLED comprises a blue-emitting OLED constructed using the following layering structure:
a 68 nm layer 23 of NPD;
a 10 nm layer 34 of Bathocuproine (2,9-dimethyl-4,7,-diphenyl-1,10-phenanthroline);
a 38 nm layer 35 of AlQ;
a 0.6 nm layer 16 of LiF; and
a 150 nm layer of 17 Aluminium.

In order to provide a NIR-emitting OLED, a layer 38 of Phosphor Technologies PTIR1070, held in a binder of Norland 65 optical adhesive, is also applied onto the light-emitting face of the flexible substrate. The phosphor layer acts to convert the blue light emitted by the OLED into infra red light at 885 nm. An infra-red edge filter 39, arranged to cut out unwanted visible wavelengths, is then optionally bonded on top of the phosphor layer. Whilst the above embodiment uses a blue-emitting OLED, a wide variety of blue emitters available. Some are polymers rather than OLEDs, and do not have to be vacuum deposited: they can simply be spun or coated onto the substrate surface. One particular such device structure is:
anode (e.g. ITO)
polymer (e.g. 500 nm thick layer)
cathode (e.g. 100 nm Calcium or Magnesium)
   where the polymer layer may be one of:
PFO Poly(9,9- dioctylfluoren-2,7,diyl), emitting at 436 nm, or
Poly-TPD Poly (N,N′-bis(4-butylphenyl)- N,N′-bis(phenyl) benzidine) emitting at 420 nm

Emission around 450 nm is preferred for blue emitter, since this is where phosphor is most sensitive to blue light.

In this as in other cases however, it will be apparent that it is not necessary that the emitter emit exclusively at this specific wavelength, but rather that it is sufficient that it emit sufficiently at a wavelength which is absorbed by the phosphor (i.e. the wavelength converting) layer. In that regard, another suitable source of emissions is in fact a nominally “green” emitting OLED. Such an emitter may be comprise:
a 68 nm layer of NPD
a 38 nmm layer of AlQ
a 0.6 nm layer of LiF
a 150 nm layer of Al

The resulting OLED emits at around 530 nm, but with broad wavelength emission. The reason this works is that the phosphor has a broad absorption region, which overlaps sufficiently with the emission spectrum of the nominally “green” OLED.

It will be apparent to those skilled in the art that by suitable choice of phosphor, emission at a selected wavelength may be obtained from wavelength shifting OLED or LEP devices which have their primary emission throughout the visible, UV or short wavelength IR spectrum, subject to the known condition that in order to achieve efficient wavelength shifting it is desirable to select materials such that the primary OLED or LEP emission is at shorter wavelength than the wavelength shifted emission from the phosphor.

LEDs have a very small emission area around their junction and, even with the use of lenses in the LED casing, the area directly illuminated by an LED is very small. OLED emission in contrast is Lambertian and emits isotropically in a 360 degree field from the whole area of the OLED. In some respects this is an advantage for a pulse oximeter light source or other medical light source, since alignment of the light source with the detector is less critical than when using the more directional LEDs. However light radiated behind and to the side of the emitter, in effect away from the patient, is wasted so far as the medical use is concerned. The arrangement can therefore be made more efficient by directing more of the light emitted by the OLED towards the patient and/or detector.

Another area of difference between OLEDs and LED emission is the width of the emission wavelength envelope. OLED emission is typically broad with, for example, a Full Width Half-Maximum (FWHM) characteristic of perhaps 100 nm. LED emission is typically sharp, with a FWHM of perhaps 20 nm. The present inventors have realised that it is nevertheless not advantageous to have a broad emission spectrum for pulse oximetry since this can introduce extra uncertainty in the saturation measurement, especially if using an empirical formula to calculate the saturation.

A further potential difficulty in using either OLEDs and LEDs is that in the case of LEDs they are available at certain wavelengths only and, similarly for OLEDs, emission at a certain wavelength is dependent on having a suitably efficient fluorescent emitter available. Wavelengths selected for use in pulse oximeters and similar purposes are therefore sometimes selected for reasons of availability rather than as being the optimum wavelength for the purpose. Hence if the emission from an OLED could be shifted from that of the currently available emitters to a more optimum one for pulse oximetry, this would be advantageous.

The present inventors have realised that it is possible to manipulate the emission of OLED devices using gratings or other structures located between the OLED and the substrate (glass or plastic) through which light is emitted, and that such devices would have use in medical light sources.

One such device comprises a thin photoresist (photosensitive polymer) layer spun onto the back of an ITO-coated substrate. The photoresist layer is then patterned, using two lasers for example, to form an interference grating with a pitch of 600 nm and a grating depth of 100 nm. On the front (ITO) side of the substrate an OLED is constructed with the following structure:
a 68 nm layer of NPD
a 38 nm layer of ALQ
a 0.6 nm layer of LiF
a 100 nm layer of Al

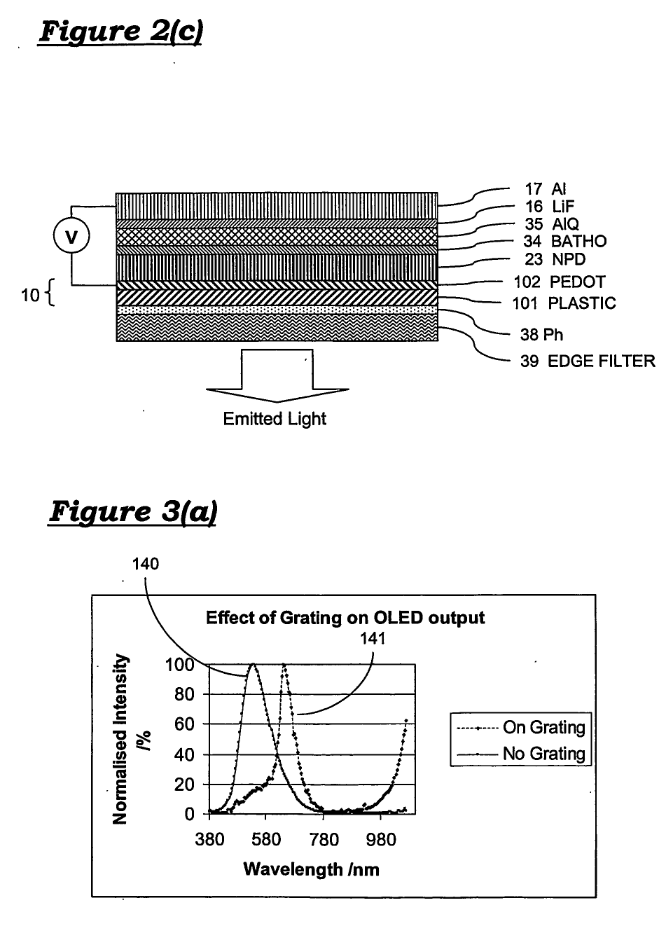

Referring now to FIG. 3(a), the resulting emission 140 is shifted 141, by the grating, towards the red spectrum: in this case the normal emission peak of 520 nm is shifted to 650 nm. In addition the FWHM of the emissions is decreased by the grating structure from 100 nm to approx 75 nm. The shift in wavelength towards the red is particularly useful since red-emitting OLEDs are inherently less efficient than green OLEDs, and the wavelength-shifted emissions at 650 nm would be ideal for applications such as pulse oximetry, without having to add dopants to the structure in order to produce a red-emitting OLED.

Even though the grating structure is on the opposite side of the substrate to the OLED in this case, it still influences the emission where it emerges from the OLED through the substrate. Such a structure increases the amount of light emerging from the substrate by extracting light which would otherwise be lost in guided modes between the glass and the OLED.

Alternative constructions of suitable grating device involve making the photoresist grating structure on top of the ITO layer—being thin the photoresist layer does not impede conductivity significantly—or constructing the photoresist grating on plain substrate and add a thin semitransparent layer of gold on top as an anode. Alternatively the grating may itself be formed from ITO or similar material.

In this context the term grating is intended to encompass single gratings, bi-gratings, multi-gratings, periodic arrays (e.g. dots or pits) whether 1-dimensional or 2-dimensional, and also quasi-periodic arrays, along with similar structures as would be apparent to the skilled person. Furthermore, although in the specific embodiment described above the grating is located between the OLED and substrate, other arrangements are possible. These include forming the grating in the substrate itself, or in a conductive layer. The grating may even be formed as part of the cathode, or in any other location in which adequate coupling can be achieved between the grating structure and the optical modes of the emitting device, including on the face upon which the emitter is formed.

Whilst the embodiments described above use semi-transparent grating structures, and referring now to FIG. 3(b), it is also possible to use structures comprising two reflective surfaces (patterned or not), the two reflective surfaces being disposed to form a micro-cavity. A micro-cavity is a Fabry-Perot cavity comprising two mirrors, in this case approximately 1000 nm apart.

A simple example of such a device comprises a thin (30 nm) layer of semi-transparent (or more generally partially transparent) gold on top of a substrate. On top of this reflector an OLED is constructed with further structure:
a 68 nm layer of NPD (N,N′-diphenyl-N,N′-bis(1-naphthyl phenyl)-1,1′-biphenyl-4,4′-diamine)
a 38 nm layer of AlQ
a layer of MgAg (as second reflector and cathode).

The OLED emission 142 from the simple cavity formed between the two reflectors is both shifted in wavelength 143 and decreased in FWHM. The peak emission wavelength for a corresponding device without the cavity is 522 nm; with the cavity structure present the peak emission is 567 nm. In addition, the FWHM decreases from approx 100 nm to 50 nm.

Referring now to FIG. 3(c), more complex device may, for example, comprise a layer structure such as:
a dielectric reflector layer
a layer of SiO₂
a layer of ITO (Indium Tin Oxide)
a 68 nm layer of NPD
a 38 nm layer of ALQ
a Mg/Ag layer (as mirror and cathode)

The spacer layer of SiO₂ is added between the dielectric reflector and the ITO layer to tune the cavity spacing to the best effect. The ITO is then sputtered on top of this dielectric layer, and the OLED constructed on top as usual by vacuum deposition.

This device exhibits a strong tuning effect, in that the position of peak emission changes at angles away from perpendicular to the device: for example peaks 145a, 145b relate to emissions at 0 degrees to the perpendicular to the plane of the cavity whilst peaks 146a, 146b are the corresponding peaks observed 30 degrees to the perpendicular to the plane of the cavity. The peak emission also splits into two peaks in each case. This means that a higher or lower wavelength emission could be engineered by careful design of the cavity, device structure, and angle. This would be useful if, for example, blue emissions were desired for a pulse oximeter to stimulate an infra-red emitting phosphor which absorbs towards the blue primarily. By shifting the emitted wavelengths by means of a micro-cavity as described above, a green-emitting OLED may be employed, the micro-cavity being arranged to shift the wavelength into the blue region, whereby to stimulate the IP-emitting phosphor to emit the required infra-red emissions. The use of green—or other coloured—source emitters may be preferred in a particular instance for reasons of cost, convenience or efficiency.

In this design a semi-transparent gold anode is used, to allow the light to escape from the device. Other designs include using a array of dots or pits to create the cavity effect and yet still allow the light to exit the device.

The above devices are described with reference to the use of light emitting layers comprising evaporated layers of low molecular mass materials. However it will be apparent to those skilled in the art that solution-processed polymeric materials such as MEH-PPV (Poly(1-Methoxy-4-(2-Ethylhexyloxy)p-phenylenevinylene)), dendrimers, and other solution-processed semiconducting and light emitting layers may be used analogously in devices comprising a grating structure, cavity structure, or both to achieve the desired result of optimising the wavelength, emission half width, and directionality of the emitted light.

The light-emitting part of the sensor may not be air-stable, and should typically therefore be encapsulated (for example for use in oximeters and other therapeutic apparatus). This is also important for protecting the skin from the substances used to construct the light sources and photo-conducting layers. A proprietary method of encapsulation can be used for this purpose. One such method is to apply one micron of parylene (poly-paraxylylene) over the whole device, followed by 150 nm of Aluminium over the face upon which the light sources and photo-conductor have been constructed. A third layer, of parylene, at one micron thick is added over the whole device. Other methods of encapsulation may also be used as would be apparent to the skilled person in the art.

For applications such as pulse oximeter sensors, it is important that a sufficient amount of light is provided to penetrate the tissue of the patient, preferably regardless of how thick the area of tissue is: for example, finger diameter varies greatly between individuals but, ideally at least, the same design of sensor should be usable on all such individuals. Thicker tissue will clearly absorb more light and decrease the magnitude of pulse signal detected, thereby reducing the signal-to-noise ratio. Since some OLED devices are less bright than many LED devices used in conventional devices, it is desirable to improve the light output (i.e. efficiency) of OLED devices for, applications such as pulse oximeters. Increasing their efficiency also has the benefit of increasing device lifetime and reducing power requirements.

In order to make emitters that have a long operational life, a large optical power output, and low power requirements, the power efficiency and external quantum efficiency of these organic electroluminescent (OEL) devices needs to be maximised.

The power efficiency of a light emitting device is the ratio of the amount of optical power emitted compared to, the energy supplied to the device and the external quantum efficiency is the ratio of the number of photons that escape from the device compared to the number of electrons supplied to the device. The power efficiency can be enhanced by minimising the electrical resistance of the device, and the external power efficiency, ηₑₓ, depends on the different factors given in the following relationship:
ηₑₓ=η_{pL}×ηₒᵤₜ×ηₛ₋ₜ×η_{rec}×η_{bal}
where
ηₑₓ=external quantum efficiency,
η_{pL}=photoluminescence efficiency,
ηₒᵤₜ=outcoupling efficiency,
ηₛ₋ₜ=singlet to triplet generation ratio,
η_{rec}=recombination efficiency of holes to electrons and
η_{ba}l=charge balance.

When current flows through an OLED, some of the charges recombine. The recombined charges either form singlet excited states or triplet excited states. In general, the ratios for the formation of singlet to triplet excited states in OLEDs are 1:4 and 3:4 respectively. The singlet excited states relax and emit light whereas, unless special measures are taken, the triplet excited states relax via a radiation-less pathway,

Incorporation of phosphorescent material in an OLED can therefore give a large increase in the optical power produced. This is achieved by generating useful light from the 75% of the generated excited states which form as triplets. The most efficient phosphorescent materials used to dope OLEDs are iridium-based organo-metallic phosphors (e.g. iridium tris-(phenylpyridine) (Ir(ppy)₃)). The results shown in Table 1 indicate a significant improvement in device performance when OLEDs are doped with phosphorescent materials are employed.

TABLE 1
Luminance Efficiency of OLED devices.

| | power efficiency of | | power efficiency of | |
|---|---|---|---|---|
| Emission Colour | fluorescent systems | | phosphorescent systems | |
| Blue | 5-8 | lm/W | 20-30 | lm/W* |
| Green | 10-15 | lm/W | 40-60 | lm/W |
| Red | 1-3 | lm/W | 4-10 | lm/W |
| White | 10-15 | lm/W | 40-60 | lm/W* |

(*indicates that these values are extrapolated)

The improved efficiency of phosphorescent OLEDs also leads to an increase in device lifetime for the red and green emitters as shown in Table 2.

TABLE 2
The lifetime of OLEDs driven at 100 cd/m²

| | Devices that harvest | Devices that harvest |
|---|---|---|
| Emission | fluorescence/hours | phosphorescence/hours |
| Blue | <5000 | <5,000 |
| Green | 70,000 | 80,000 |
| Red | 40,000 | 50,000 |

A difficulty with phosphorescent (triplet) emission however is that the decay lifetime is much longer than that for singlet emission. This lifetime is sufficiently long that it may interfere with the operation of, for example, a pulse oximeter, which is typically driven in a pulsed fashion at high frequency. The emission lifetime of the triplet emitter must therefore be less than the repetition rate of the pulse oximeter device.

The detector may additionally be gated to synchronise with the light emissions to enhance detection and reduce effects of background light, whether from preceding flashes or from other sources. This also allows the emitters to be powered down between “bursts” of flashes synchronised with the patient's pulse peak and trough periods, which has the added benefit of reducing power dissipation into the patient's body.

For oximetry and similar applications it is necessary to sample the optical density of tissue at least at the maximum and minimum point of each pulse and hence at least twice per pulse. This normally achieved by sampling much more frequently (for example, 20-50 times per patient pulse) and using some kind of curve fit or other appropriate method to pick out the peak and trough. The present inventors have also realised that it is possible to use an oximeter sensor in conjunction with another probe, for example an ECG or other device which can determine pulse timings. The patient pulse timing information received from the probe may then be used to reduce the number of samples taken. In an extreme case sampling may be reduced to just two samples per pulse, though in practice it may be more practical to reduce sampling from the entire duration of each pulse to two sub-regions associated with the peak and trough identified by the ECG. That sets an upper practical limit on t₂ based on lowest pulse rate to be analysed, and which could be in the region of 600 ms.

The restriction on oximeter repeat interval then arises because two light sources (red and infra-red) are required. Power consumption, and more importantly power emissions, can be optimised by ensuring the OLEDs are powered down during a significant fraction of the sampling cycle.

It is therefore desirable to emit narrow (e.g. 1 ms) flashes of light from each of at least two light sources emitting at distinct wavelengths, the successive flashes from alternate sources being timed widely apart (e.g. 20 ms) relative to the individual flash duration. In using triplet emissions it is therefore necessary to consider particularly the effect of the longer emission duration, in determining appropriate timings: emissions from a first emitter must have substantially died away before the next emitter flash is initiated so for triplet emitters allowance must be made for adequate decay of the luminescence between “flashes”.

The present inventors have found that for an emitter with an emission lifetime, t₁, for use in a pulse oximeter with repeat period, t₂, the triplet emitter may usefully be activated during a time, t₃, characterised by the relationships:
t₁≦t₃/2
to allow at least one sample at each wavelength (red and infra-red for the oximeter application) per period. In general should be made short enough to allow alternate colour samples and furthermore, the emission lifetime should be much shorter, maximising peak emission intensity—to allow good detection—whilst minimising overall power dissipation.
t₃≦¾ t₂
where a typical repeat period, t₂, for a pulse oximeter application is less than or equal to approximately 25 ms.

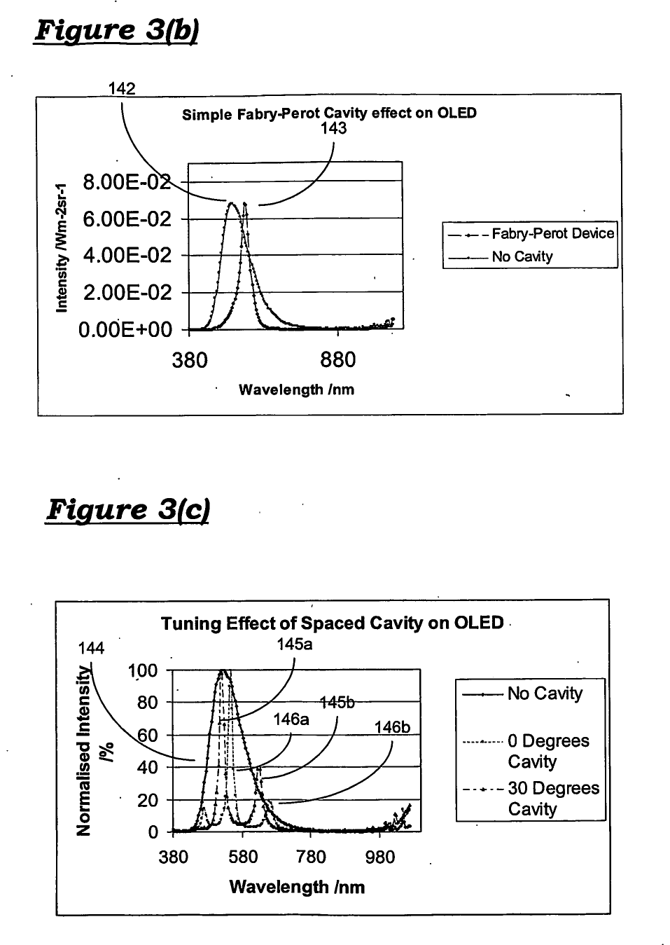

Several triplet emitter systems have been identified which fulfil the above timing criteria. In particular, and referring now to FIG. 4, a first example device uses an Iridium organometallic complex Ir(ppy)3, with a layering structure of:
a layer 102a of ITO (as anode)
a 40 nm layer 23 of NPD (N,N′-diphenyl-N,N′-bis(1-naphthyl phenyl)-1,1′-biphenyl4,4′-diamine)
a 20 nm layer 130 of Ir(ppy)3 in CBP (4,4′-bis-(carbazol-9-yl) biphenyl)
a 0.6 nm 131 layer of BCP (Bathocuproine)
a 20 nm layer 35 of AlQ3 (Aluminium tris(8-hydroxyquinoline))
a layer 132 of MgAg (as cathode)

The triplet lifetime on this system is approximately 500 ns. The doping level of the Ir(ppy)3 is 6% with respect to the CBP. This particular device will emit at green wavelengths. There are, however, variations to the ligand which enable such a system to emit at red wavelengths. For example, by doping CBP at 7% with the molecule Ir(btp)3 enables triplet emission at 617 nm.

Another suitable embodiment (not shown) uses a platinum metal in a porphyrin ligand complex. This has a longer triplet lifetime of approx 100 μs. One suitable device structure is as follows:
a layer of ITO (as anode)
a 0.6 nm layer of BCP
a 45 nm layer of NPD
a 40 nm layer of PtOEP/AlQ3 (where PtOEp is Platinum octaethylporphyrin)
a 20 nm layer of AlQ3
a layer of MgAg (as cathode)
   where PtOEp is Platinum octaethylporphyrin.

Use of PtOEP will cause light to be emitted at 650 nm, which would be useful for pulse oximeter light sources.

A further embodiment (not shown) which produces the desired benefits for use in pulse oximeter light sources is one which uses Ir(ppy)3 (i.e. fac-tris-(2-phenylpyridine) Iridium )as a sensitizer for a dye emitting at the desired wavelength. In the situation with a fluorescent dye in a host material, it is desirable to transfer triplet excitons in the host material to the fluorescent dye. This is made easier by adding a phosphorescent dopant which allows triplet states in the host to be transferred to the dye via singlet and triplet states in the dopant. One example of such a system is one in which the CBP host is doped with both DCM laser dye at 0.2% and Ir(ppy)3 at 8%. The result is nearly complete energy transfer from Ir(ppy)3 to DCM. This system is again useful for pulse oximeter light sources, and other applications requiring red emissions, as it would allow a high efficiency red OLED to be made.

A still further arrangement (not shown) by which red triplet emission can be achieved is using Eu3+ ions in a ligand complex. Rare earth complexes are characterised by efficient energy transfer between ligand singlet and triplet states and thence to the metal ion excited state. For this reason rare earth complexes are expected to be highly efficient emitters in OLEDs. The following examples may be constructed using conventional vacuum deposition. The rare earth complex used is europium (dibenzoylmethanato)3(bathophenanthroline) [Eu(DBM)3bath]. A typical double layer device structure is as follows:
a layer of ITO
a 30 nm layer of NPD
a 80 nm layer of Eu(DBM)3bath
a layer of MgAg (Magnesium—Silver)

Alternatively a triple layer device may be constructed:
a layer of ITO
a 30 nm layer of NPD
a layer of NPD:Eu(DBM)3bath
a 50 nm layer of Eu(DBM)3bath
a layer of MgAg

The concentration of Eu(DBM)3bath in the NPD host is 2%.

Such devices emit at approximately 620 nm, and, although they have a longer triplet decay time than the other devices described above, they are still within the limits for use in pulse oximetry and related applications, with a lifetime of approx 1 ms.

To complement the flexible light source, a flexible photo-detector may be provided by forming a photo-detector upon a flexible substrate in a fashion similar to that for creating the flexible OLED. In the applications proposed below however, and unlike their conventional use in devices such as photocopiers and laser printers, the photo-detector is arranged to detect light in the near infra-red (NIR) to red region of the spectrum.

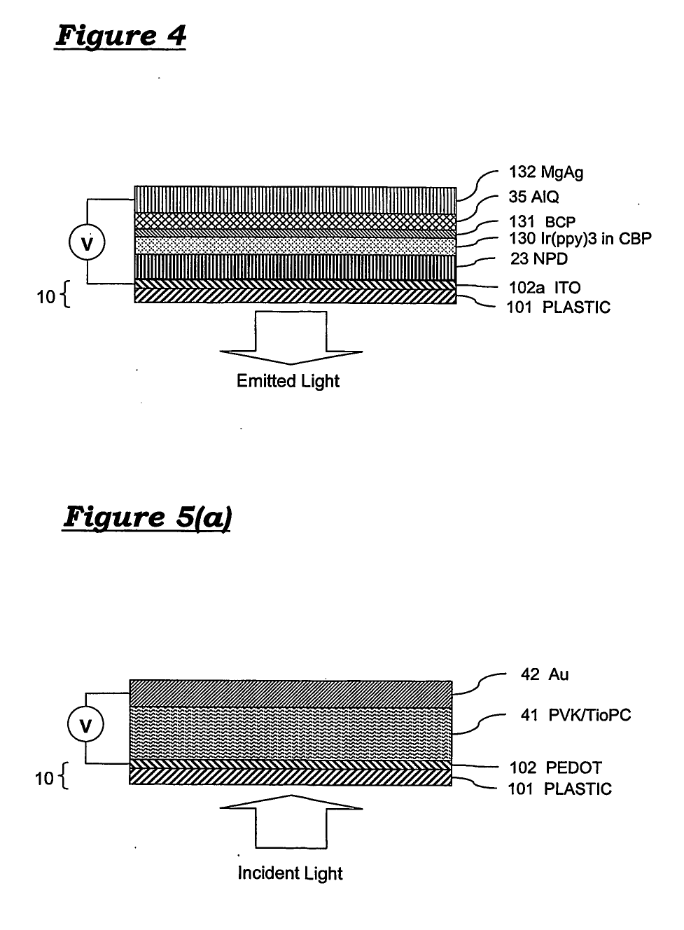

Referring now to FIG. 5(a), a suitable flexible photo-detector comprises a first layer 41 formed from an organic photo-conductor made, in this embodiment, from a solution of Poly Vinyl Carbazole (PVK) in Dichlorobenzene (DCB) at a 10% concentration. Into this solution is mixed a finely ground sample of Titanyl Phthalocyanine (TiOPC) in the ratio 3:1 (PVK:TiOPC). The resulting mixture is then spun onto the plastic substrate at 2000 rpm for 30 seconds to give a layer in the order of 3-5 μm thick. A 100 nm layer 42 of gold acts as cathode.

Preferably the photo-detector is formed on the substrate before formation of the OLEDs as described above.

Whilst organic photoconductor materials—such as those having phthalocyanine layers bound in polymers as described above—may be used to make a fully flexible, sensitive light detector element for medical sensors including pulse oximeters, it has been found that photovoltaic detectors have certain advantages over the photoconductive sensors (organic and inorganic) currently used in pulse oximetry. Photovoltaic detectors are less prone to picking up excessive noise which decreases the signal-noise ratio. Their response is also more linear with light intensity; sensitivity of photoconductive sensors falls in the presence of strong background light. The conventional algorithms used to convert signals to the saturation value assume that detectors have a linear response, so a more complex correction function must be used to calculate the saturation where the sensor response is significantly non-linear.

A photovoltaic light detector is commonly constructed by creating a P (positive) and N (negative) junction. These materials may be doped crystalline silicon, other inorganic materials or organic semiconductor layers. When light is incident upon the junction charge separation occurs and a voltage is induced. This signal may then be detected in either a current or a voltage mode.

Use of organic photovoltaics in the detector has the following benefits over use of photoconductor detectors: it allows easier device preparation and large area fabrication is straightforward; organic photovoltaics may be more flexible; they use low-toxicity materials; and they are efficient in coupling of light due to the relatively low refractive index.

It is of course important to choose a photovoltaic system which will respond to the infra-red and red wavelengths used in a pulse oximeter device and phthalocyanine-based or perylene based photovoltaic devices are found to respond at the red and infra-red wavelengths required for pulse oximetry.

Referring now to FIG. 5(b), one example of a photovoltaic detector device layering scheme which may be used in a medical sensor such as a organic pulse oximeter is:
a layer 102a of ITO (as anode)
a 32 nm layer 120 of PEDOT:PSS (poly (3,4,ethylenedioxythiophene): polystyrenesulphonic acid)
a 20 nm layer 121 of CuPC (Copper Phthalocyanine)
a 40 nm layer 122 of C₆₀
a 12 nm layer 123 of Bathocuproine (BCP)
a 100 nm layer 17 of Al (as cathode)

The CuPC acts as a donor layer and the fullerene (C₆₀) as an acceptor layer. The purpose of the BCP is to transport electrons from the cathode to the acceptor layer while preventing excitons from the donor layer from recombining at the cathode.

PEDOT:PSS is a high work function hole injection layer deposited by spin coating onto cleaned ITO. The other materials in the device are deposited by vacuum deposition.

Referring now to FIG. 5(c), this system can be further improved by having a more gradual boundary between donor and acceptor layers instead of a single sharp junction. For example a layer structure of;
a layer 102a of ITO (as anode)
a 3.5 nm layer 121 of CuPC
a 16.7 nm layer 121a of 75% CuPC, 25% C₆₀
a 16.7 nm layer 121b of 50% CuPC
a 16.7 nm layer 121c of 25% CuPC
a 5 nm layer of 122 C₆₀
a 12 nm layer of 123 BCP (Bathocuproine)
a 100 nm layer 17 of Al (as cathode)

Such a structure gives approx twice the efficiency of a double layered structure. It is thought to bring this improvement by using the composition gradient to drive charges more easily towards the relevant electrode.

In another embodiment shown in FIG. 5(d) a two-layer system is used utilising an evaporated perylene layer and a spin-coated MEH-PPV layer. Both layers produce excitons under illumination, and the excitons appear to be dissociated into electrons and holes for conduction at the boundary between the layers. The following device structure is used:
a layer 102a of ITO (as anode)
a 10 nm layer 124 of PpyEl (perylene bis(pyridyl ethylimide))
a 30 nm layer 125 of M3EH-PPV (poly (2,5-dimethoxy-1,4-phenylene-1,2-ethenylene-2-methoxy-5(2-ethylhexyloxy)-1,4-phenylene-1,2-ethenylene)
a 100 nm layer 42 of Au (as cathode)

Alternatively a device, illustrated in FIG. 5(e), may be fabricated from a two-layer CuPC/perylene system. For example:
a layer 102a of ITO (as anode)
a 30 nm layer 126 of CuPC (Copper Phthalocyanine)
a 50 nm layer 127 of PV (Perylene tetracarboxylic acid bis-benzimidazole)
a layer 43 of Ag (as cathode)

Whilst only three specific layering arrangements have been described in detail, it will be apparent that other layering arrangements may be used in their place, as would be apparent to the person skilled in the art.

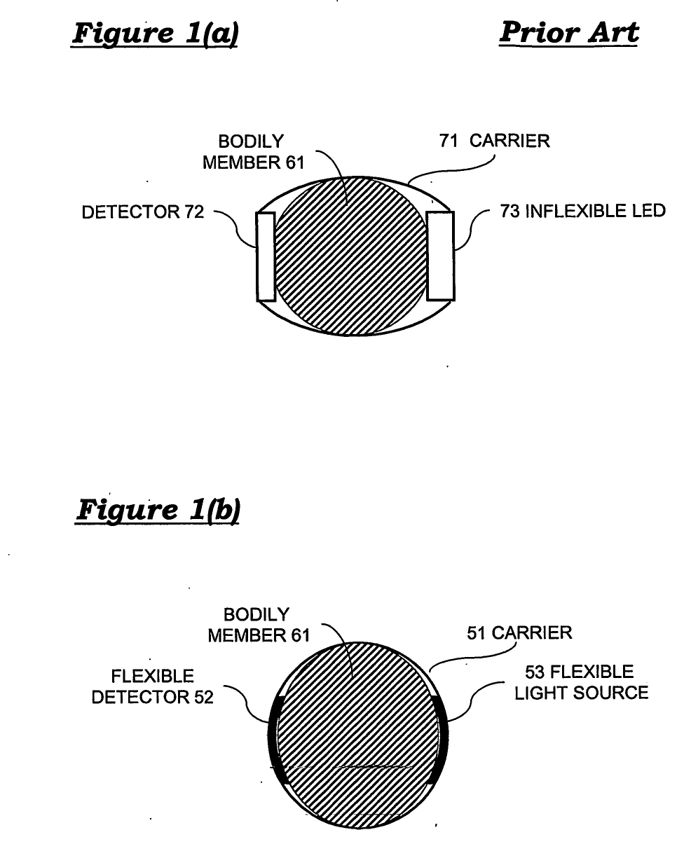

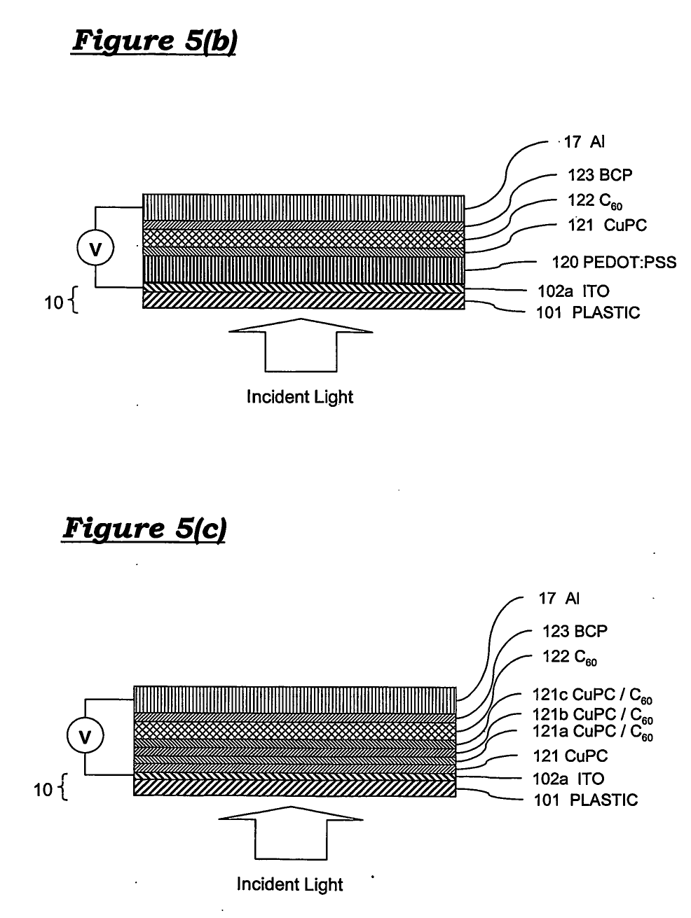

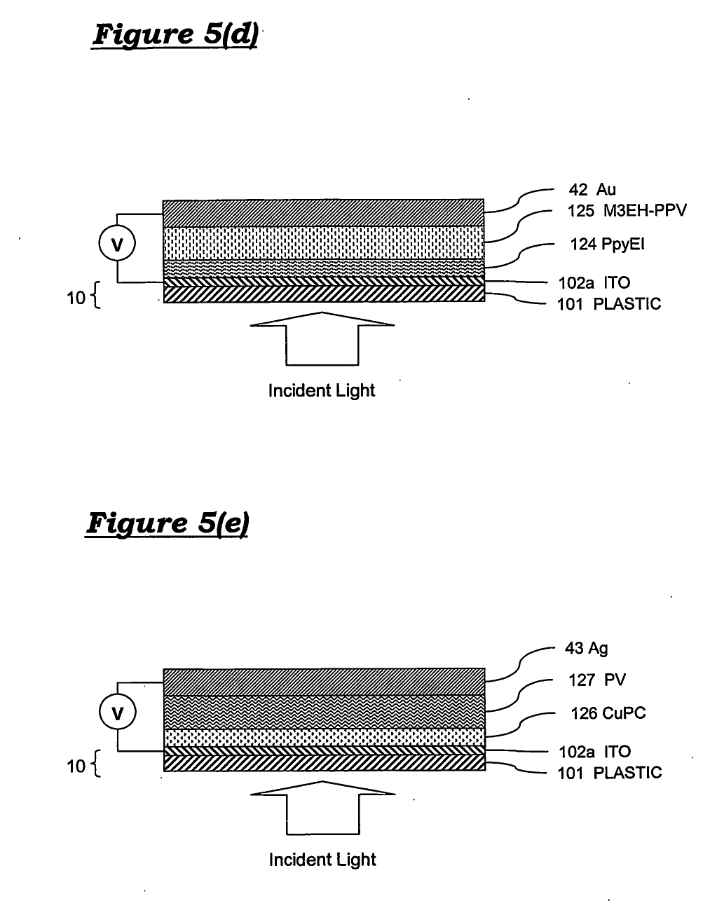

Referring now to FIGS. 1 (b), 6(a-b), and 7, a medical sensor, for example a pulse oximeter 50, may be constructed making use of such flexible OLEDs and photo-detectors. In particular, the pulse oximeter may comprise a flexible carrier strip 51 to which are attached a pair of OLEDs emitting at different wavelengths. In particular a first OLED 53 emits light in the red part of the spectrum, whilst a second OLED 54 emits in the infra-red part of the spectrum. A photo-detector 52, such as one of those described above, is located on the carrier strip such that, when the oximeter is wrapped around a bodily part 61 (for example finger or toe) to be monitored, light emitted from each OLED is received by the photo-detector through the bodily part. The photo-detector and OLEDs are powered, controlled, and monitored via electrical connecting wires 55 coupled to a control mechanism 57. Suitable mechanisms are known in the art. Many other drive schemes and analysis functions exist which would be suitable for use in conjunction with this pulse oximeter sensor.

As has been noted above, OLEDs and photo-detectors may be formed upon flexible substrates. It is therefore possible (though not essential) to form both of the OLEDs and the photo-detectors on a single substrate which forms the flexible carrier strap. This simplifies manufacture by removing steps associated with attaching separately manufactured light sources and detectors to a separate carrier strap as in known sensors. Clearly in the present arrangement, all necessary electrical connections may also be formed upon the same substrate as part of the same manufacturing process.

Nevertheless embodiments may be manufactured comprising flexible devices formed on one or more areas of substrate attached to a distinct support member. The support member may, for example, comprise an elastic or other fixing means. The fixing means in each case may be arranged to limit the tightness experienced by the patient when in use.

Whilst the present embodiment shows only a single detector 52 sensitive to the emission wavelengths of both light sources 53, 54, alternative embodiments clearly include those having multiple detectors, each sensitive to emissions from respective light sources.

The carrier strip 51 may be fixed around the patient by any of a number of attachment means including in particular hook-and-loop means 56a, 56b. Hook-and-loop means is particularly suitable for this arrangement since the flexibility of the OLEDs and photo-detector allow the carrier strip to follow the contours of the bodily part much more closely than do the rigid components of known oximeters. As a result there is much less likelihood of slippage around or off of the patient's digit or limb. The carrier strip itself 51 may be of a stretchable material to facilitate attachment and allow for some variation in patient sizes. The use of hook-and-loop style fastenings (or indeed other re-usable fastening means including “poppers”) also facilitates repeated removal and reattachment of the oximeter without loss of fastening strength or functionality.

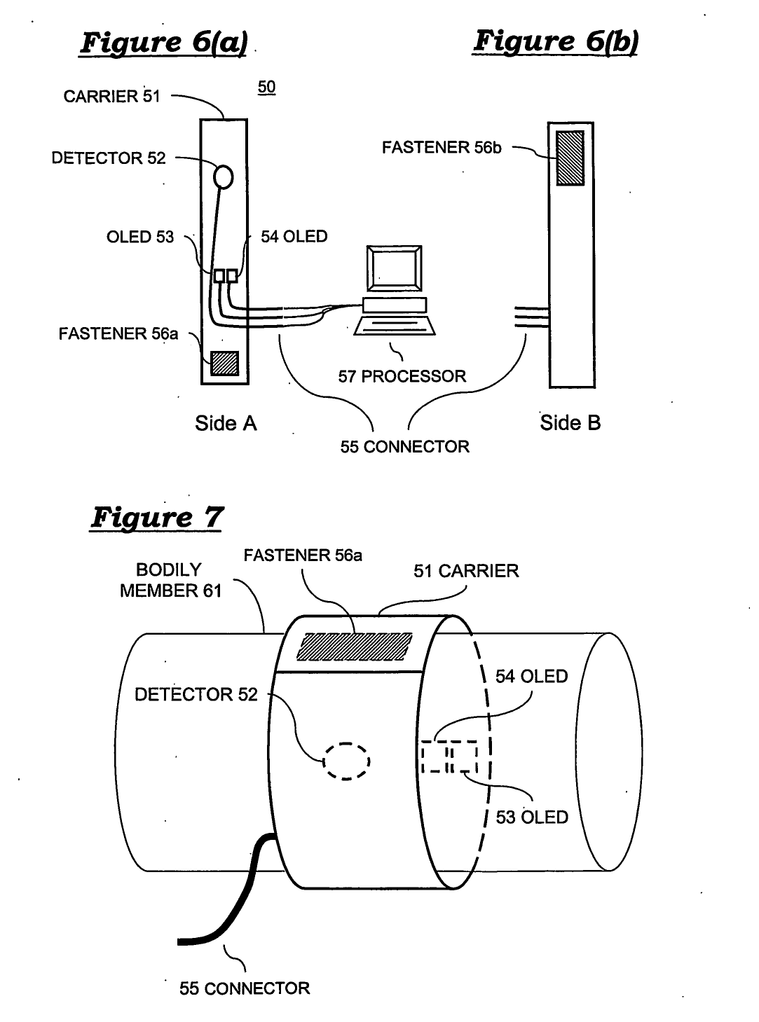

In a further embodiment, shown in FIG. 8, the substrate forming the strap itself may be formed to provide the attachment means. One or more slots 96a may be provided in one end of the strap whilst the other end is narrowed and provided with barbs 96b. In operation, the strap may be passed around the patient and the barbed end slid through one or other of the slits and gently tightened sufficiently to retain the strap around the patient. Clearly, two or more pairs of slots and barbed inserts may be used where appropriate, especially for larger devices.

This form of attachment obviates having to attach additional components to the strap to provide the attachment means. Instead the straps may be simply cut to shape from a sheet or roll during manufacture in a simple, continuous operation.

In another embodiment, a portion of the strap is pre-coated with an adhesive so that, in operation, that portion of the strap may be stuck to the outside face of the strap when placed around a patient. This avoids applying the adhesive directly to the patient.

Although the description above has been directed to a pulse oximeter, the techniques involved may of course be applied to a wider range of devices and applications.

In particular, the number of OLEDs in a given device may be increased to three or more, each emitting at a distinct wavelength so as to provide for different sensors adapted to detect other patient characteristics.

It is possible then to measure the concentration of other components in the blood (for example carbon monoxide or bilirubin) by using a third wavelength light source and solving three simultaneous equations using, for example, the technique described in patent U.S. Pat. No. 4,167,331. Detection of both CO and bilirubin relies on a device which emits at 668 nm. Such a source may be based upon Poly ([9,9-dihexyl-2,7-bis(1-cyanovinylene) fluorenylene]-alt-co-[2,5-bis(N,N′-diphenylamino)-1,4-phenylene]) (“Poly-CFD”). This compound is available from HW Sands Ltd as Catalogue number OHA2212. The OLED has the following structure:
anode (e.g. ITO/PEDOT);
polymer (Poly-CFD as above) (e.g. 500 nm)
cathode (e.g. calcium/Magnesium) (100 nm)

The resulting OLED emits at approximately 668 nm. By employing such an emitter in conjunction with, for example, two OLEDS as for the pulse oximeters 50 described above, it is possible to provide a detector for carbon monoxide (CO) levels in the bloodstream.

A further embodiment provides a sensor for cardiac output measurement using a well-known technique involving injecting dye into a site. By measuring and comparing the dye concentration upstream and downstream of the injection site, cardiac output or flow may be determined. This is known as Fick's Principle.

Some such dyes include Methylene Blue, which absorbs light at 668 nm. By constructing a sensor comprising two OLEDS as in the pulse oximeter in combination with a third OLED emitting at 668 nm, the concentration of Methylene Blue, and hence cardiac flow, may be determined.

To prevent stray incident light from other sources affecting the photodetector, a suitably light-proof layer may be provided around the back of the light sources and/or detector. The layer may take any suitable form to block incident light from the rear of the OLED and detector: for example as a light-proof layer deposited on the back of the OLED, or as a separate physical member attached to the OLED, or a separate physical member merely loosely wrapped around the OLED while in use. The light-proofing should be sufficient at least to block wavelengths to which the detector is sensitive.

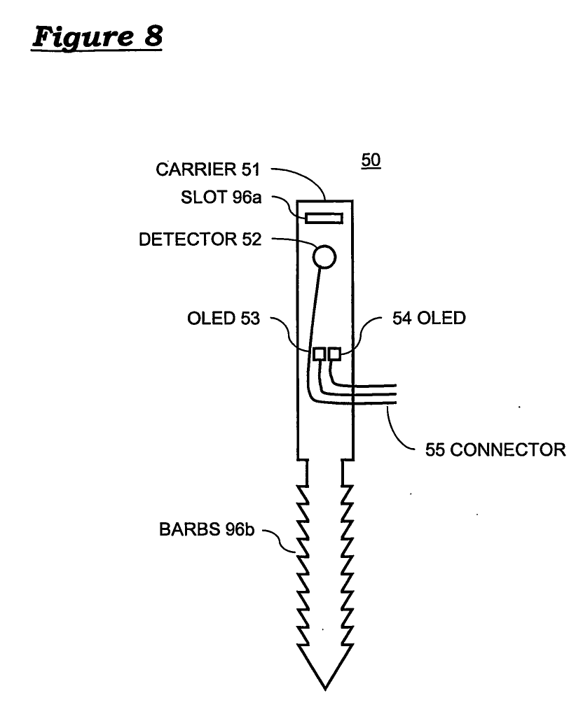

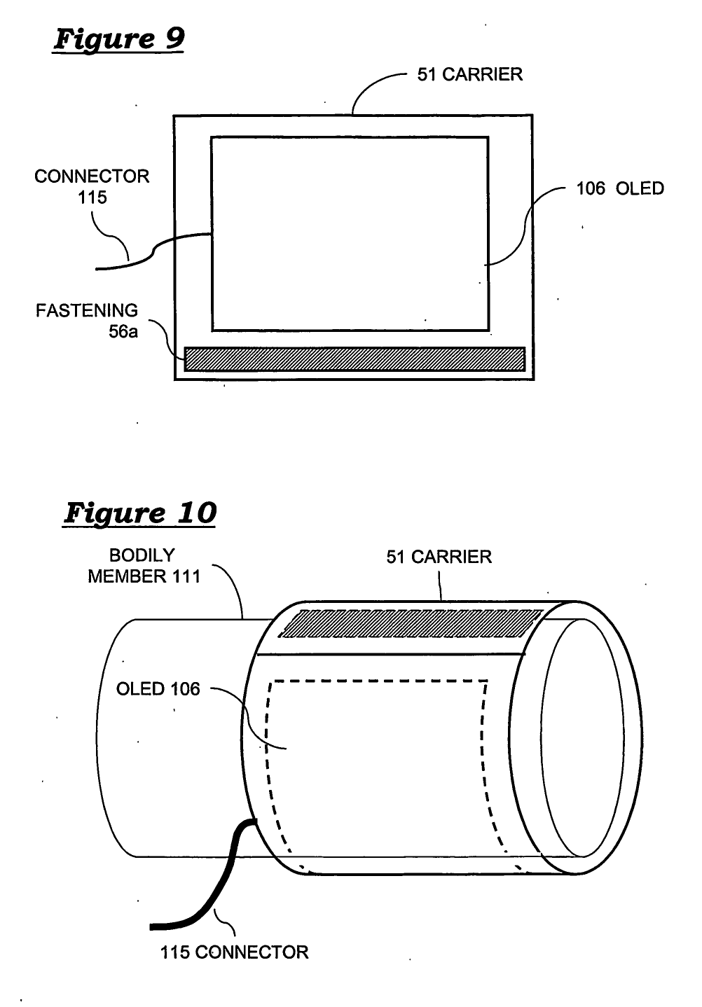

Referring now to FIGS. 9 and 10, in another embodiment, a flexible light source is provided to illuminate a portion of a body with light of a predetermined wavelength, the chosen wavelength having a therapeutic value. The flexible light source may be in the form of an OLED 106. In such applications the area of the OLED will typically be much larger than that employed in, for example, the pulse oximeter. This is because it will often be appropriate to illuminate a largish portion of the patient's body. However, for highly localised treatment, smaller light sources could of course be employed. Flexible light sources may therefore be readily manufactured in any size to suit different treatments.

Employing a flexible light source has the advantage that, instead of requiring the patient to remain stationary in the vicinity of a large and unwieldy light source, the new device—being lightweight, flexible, and portable—may be rolled or otherwise applied relatively closely over or around a bodily member 111 and can be readily carried around by the patient during treatment.

One particular such application is for UV Phototherapy for skin conditions including, but not limited to, psoriasis. The inventors have noted that Poly[(9,9-dioctylfluoren-2,7-diyl)-alt-co-(2,2′-bipyridin-6,6′-diyl)] (PFO-BD) is a UV OLED emitter (available from HW Sands Ltd catalogue number OPA3191) emits at 369 and 392 nm when cast from solution. It is therefore possible to construct a flexible OLED having the following structure:
anode (e.g. ITO/PEDOT)
PFO-BD (e.g. 500 nm)
cathode (e.g. Calcium or Magnesium 100 nm)

To prevent leakage of UV light in unwanted directions, a light-proof layer, optionally a reflective layer, may be provided around the back of the light source. The layer may take any suitable form to block emissions to the rear of the OLED: for example as a UV light-proof layer deposited on the back of the OLED, or as a separate physical member attached to the OLED, or a separate physical member merely loosely wrapped around the OLED while in use.

By providing such a flexible light source which may be wrapped relatively closely around only the affected area of the body, and which mitigates stray emissions not required for therapy, the risk of damage to the eyes from stray UV light from the light source may be significantly reduced. By being able to locate the light source substantially uniformly closely around the bodily part, it is also possible to consistently obtain more even coverage to the whole of an affected area than may be possible using conventional UV lamps positioned more remotely over or around the affected area.

Turning now to applications in photodynamic therapy, one dye used is Photofrin which absorbs light at 630 nm. A red-emitting OLED emitting at around 630nm used as illuminator therefore emits at an appropriate wavelength to effect treatment. The DCM-doped OLED described above in connection with the pulse oximeter embodiment is one such OLED which may also be used for photodynamic therapy in conjunction with Photofrin.

Other dyes which may be used in photodynamic therapy include, for example, benzoporphyrin derivatives (BPD) which absorb at 680 nm. In this case a deep red emitter (around 668 nm) is required, such as that described above.

These embodiments can enable greater penetration of light to tumour sites by virtue of their wrap-around design which enables close proximity illumination and light penetration from all angles around the tumour site.

A further benefit of such medical light sources is that OLEDs emit over a relatively narrow spectrum compared to conventional lamps used for therapy. Use of OLEDs as light sources therefore helps mitigate the levels of undesirable light emissions directed to the affected area during treatment. In particular, incidental infra-red emissions may be reduced compared with known light sources. This is beneficial to the patient since excessive infra-red exposure can damage otherwise healthy tissue.

A further feature enabled through use of OLEDs rather than LEDs is that OLEDs offer a substantially 180 degree angle of illumination, compared to the narrower emission angle associated with LEDs. As a result the precise alignment on the patient of devices using OLEDs is less critical and this in itself acts to mitigate the impact of the penumbra effect in the monitoring sensors.

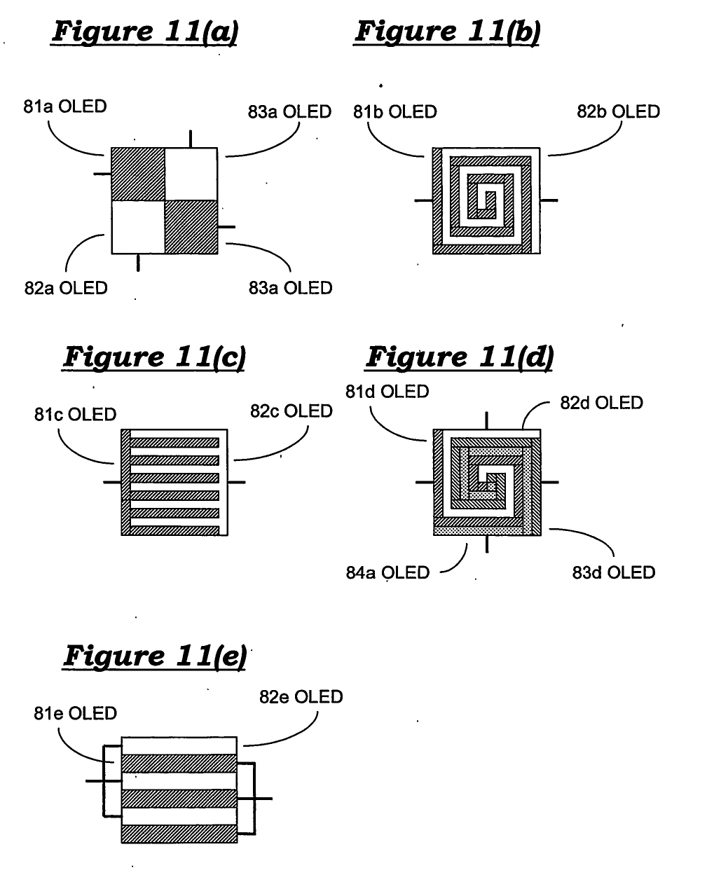

However the penumbra effect may be further mitigated by tailoring the shape of the OLEDs in the sensor so that their areas of emission are substantially interleaved in such a way that, for practical purposes, they effectively emit light over either very closely situated areas or, preferably, substantially co-extensive areas by means of, for example, chequerboarded, interleaved, or spirally interleaved arrangements of OLEDs. This can be achieved by any one of many layouts each comprising two or more OLEDs, the OLEDs being selected to emit at one of two or more respective wavelengths. Examples of such layouts are shown in FIGS. 11(a-e), which illustrate respectively:
a chequerboard arrangement of two wavelengths employing four OLEDs 81a- 84a;
a spiral arrangement of two wavelengths using two OLEDs 81b-82b;
an interleaved comb arrangement of two wavelengths using two OLEDs 81c-82c;
a spiral arrangement of four wavelengths using four OLEDs 81d-84d; and
an arrangement of two wavelengths employing six OLEDs 81e-86e, three of each wavelength.

Many other configurations are possible as would be apparent to the person skilled in the art. Mitigation of the penumbra effect makes precise alignment of the device on the patient less critical and hence more reliable and less time-consuming.

This flexibility in topography of the OLEDs is further enhanced by being able to form a single wavelength-emitting OLED in multiple, disjoint areas, and coupling together electrically emitters of the same wavelength to allow them to be operated as a single OLED. An example of one such suitable arrangement is shown in FIG. 11(a) when OLED 81(a) is coupled to OLED 83(a), OLED 82(a) is coupled to OLED 84(a). FIG. 11(e) shows an arrangement of two groups of three coupled OLEDs.

Any range or device value given herein may be extended or altered without losing the effect sought, as will be apparent to the skilled person for an understanding of the teachings herein.

1. A medical sensor comprising one or more flexible light emitting diodes formed upon respective regions of flexible substrate and one or more flexible photo-detectors formed upon respective regions of flexible substrate.

2. A medical sensor according to claim 1 in which the flexible light emitting diodes are formed upon a single flexible substrate.

3. A medical sensor according to claim 1 arranged to be sufficiently flexible to permit the light source, in operation, to conform to a portion of the body of a patient to which light from the light source is to be applied.

4. A medical sensor according to claim 1 in which the flexible light emitting source comprises an organic light emitting diode.

5. A medical sensor according to claim 1 in which the flexible light emitting diode emits light at a wavelength suitable for diagnosis or therapy of a medical condition of the human or animal body.

6. A medical sensor according to claim 1 in which the flexible light emitting diode emits light in the red to infra-red region of the spectrum.

7. A medical sensor according to claim 1 in which the flexible light emitting diode emits light in the near infra-red region of the spectrum.

8. A medical sensor according to claim 1 in which the flexible light emitting diode emits light in a non-visible region of the spectrum.

9. A medical sensor according to claim 1 comprising a plurality of flexible light emitting diodes arranged to emit light at mutually distinct wavelengths.

10. A medical sensor according to claim 1 comprising at least two light emitting diodes arranged to emit at mutually distinct wavelengths, the light emitting diodes being arranged such that light at those distinct wavelengths is emitted substantially evenly across the sum of the areas defined by the light emitting diodes emitting at those wavelengths.

11. (canceled)

12. A medical sensor according to claim 1 comprising a strap comprising attachment means for attachment of the medical light source around or to a patient's body.

13. A medical sensor according to claim 12 in which the flexible substrate forms the strap.

14. A medical sensor according to claim 12 in which the attachment means is one of hook-and-loop means, barb-and-slot means, and self-adhesive means.

15. A medical sensor according to claim 1 in which the light emitting diode comprises a triplet emitter.

16. A medical sensor according to claim 1 in which the light emitting diode comprises one or more components arranged to wavelength-shift light emitted within the light source from a first wavelength to a second wavelength.

17. A medical sensor according to claim 16 comprising a fluorescent emitter and in which wavelength-shifting is at least partially achieved by means of a fluorescent emitter.

18. A medical sensor according to claim 16 comprising a wavelength-shifting grating and in which wavelength-shifting is at least partially achieved by means of the wavelength-shifting grating.

19. A medical sensor according to claim 16 comprising a micro-cavity and in which wavelength-shifting is at least partially achieved by means of the micro-cavity.

20. A medical sensor according to claim 19 in which the second wavelength is determined by tuning of the micro-cavity.

21. A medical sensor according to claim 20 in which the micro cavity is tuned to emit light at a third wavelength substantially perpendicular to the plane of the light emitting diode.

22. (canceled)

23. A medical sensor according to claim 1 in which at least one of the one or more flexible photodetectors is arranged so as, in operation, to detect light emitted by at least one of the flexible light emitting diodes.

24. A medical sensor according to claim 23 comprising two or more flexible light emitting diodes arranged to emit light on a time-interleaved basis.

25. A medical sensor according to claim 1 comprising a plurality of the medical light sources arranged, in operation, to emit light at wavelengths suitable for diagnosis of levels of at least one of oxygen, carbon monoxide, and bilirubin in a human or animal body.

26. A medical sensor according to claim 1 in which the light detector is an organic photovoltaic detector.

27. A method of operating a medical sensor according to claim 15 in a pulsed mode having a predetermined pulse period, such that the triplet emitter is activated for a period calculated to ensure that emissions fall to acceptable levels before a subsequent light pulse is emitted.

28. A method according to claim 27 in which the predetermined pulse period is less than or equal to 25 ms.

29. A method of operating a medical sensor according to claim 1 in a pulsed mode, timing of emitted light pulses being determined responsive to an indication of the pulse timing of a patient to which the sensor is applied.

30. (canceled)

31. (canceled)

32. An organic light emitting diode arrangement comprising an organic light emitting diode arranged to emit light in the blue region of the spectrum and a wavelength-converting layer arranged to convert blue emissions from the organic light emitting diode to emissions in the infra-red region of the spectrum.

33. An organic light emitting diode arrangement according to claim 32 in which the wavelength-converting layer comprises a phosphor based compound.

34. An organic light emitting diode arrangement according to claim 32 in which the wavelength-converting layer comprises an infra-red edge filter.

35. (canceled)

36. (canceled)
